(19) United States
(12) Patent Application Publication
(10) Pub. No.: US 2022/0211390 A1
(43) Pub. Date: Jul. 7, 2022

(54) MEDICAL TECHNOLOGY POSITIONING/ALIGNMENT DEVICE WITH GUIDE TEMPLATE AND OPTICAL MARKING DEVICE

(71) Applicant: Aesculap AG, Tuttlingen (DE)

(72) Inventor: Vincent Marie, Engen (DE)

(21) Appl. No.: 17/610,905

(22) PCT Filed: May 15, 2020
(86) PCT No.: PCT/EP2020/063657

§ 371 Date: Nov. 12, 2021

(30) Foreign Application Priority Data
May 16, 2019 (DE) 10 2019 112 898.6

(51) Int. Cl. A61B 17/15; A61F 2/46; A61B 34/10; A61B 17/17

(52) U.S. Cl. CPC A61B 17/157; A61B 17/155; A61B 2034/107; A61B 34/10; A61B 17/1764; A61F 2/4657

(57) ABSTRACT

A medical technology device for positioning and/or orientating an object relative to a patient while directly visualizing on the patient geometric information that is object-specific and relates to the object's position and/or orientation. The device includes a template that co-operates with the object to be positioned and/or to be oriented, and an optical marking unit that is coupled to the template in a specific position, for visualizing the object-specific geometric information.

### CROSS-REFERENCE TO RELATED APPLICATIONS

This application is the United States national phase entry of International Application No. PCT/EP2020/063657, filed May 15, 2020, and claims the benefit of priority of German Application No. 10 2019 112 898.6, filed May 16, 2019. The contents of International Application No. PCT/EP2020/063657 and German Application No. 10 2019 112 898.6 are incorporated by reference herein in their entireties.

### FIELD

The present invention relates to a medical device for positioning and/or orienting a medical instrument for endoprosthetic care or an implant relative to a patient by directly visualizing/projecting, at or on the patient, geometric information specific to the instrument or implant and relating to its positioning and/or orientation.

### BACKGROUND

In many surgical applications, in particular musculoskeletal applications such as orthopedics, a surgical/medical instrument such as a bone saw, drill, milling cutter, etc., or implant has to be oriented relative to the patient at various structures and/or points of orientation such as bones. In this context, it is currently known to first manually align the instrument or implant and to subsequently check the alignment with a mechanical auxiliary device, such as extra-medullary alignment rods or plates for checking resection thicknesses. In this process, visual assessment of the situation at hand by the surgeon plays a decisive role, so that the quality of the alignment depends to a large extent on the experience and care of the surgeon.

A common error in alignment of instruments/implants relative to the patient/patient bone based on visual estimation is to misjudge angles and/or parallels between two objects (instrument versus bone) even when the two objects are fully visible, or to create parallax errors due to perspectives or projections perceived in wrong directions or on wrapped and/or uneven surfaces.

However, visual assessment for the alignment of surgical/medical instruments and/or implants relative to the patient/patient bone is also used in situations where structures involved in the alignment are partly not directly visible to the surgeon but are located, for example, on a bone surface that is covered by tissue and is not visible or only partly visible. A problem that often arises in this case is again often a generation of a parallax error due to an incorrect detection of perspectives or projections, for example as a result of directional errors.

The aforementioned problem occurs in particular frequently during TKA operations (total end prostheses) during alignment of incision guides for proximal tibial incisions or distal femoral incisions. In manual surgery without the use of a navigation system, such incision guides are positioned and aligned using internal or, in the case of the tibia, external alignment systems. Internal alignment systems are usually referenced relative to the bone marrow and are not visible. With external alignment systems, there are some additional rules of thumb. A space of two to three finger widths between the alignment system and the patient's bone should provide good alignment. However, all of the above solutions include the problem of parallax errors and poor perspective.

To date, there is no system on the market with which alignment axes of surgical instruments or implants, such as in particular instruments for surgery, can be displayed and/or embodied on the patient, in particular on the patient's skin, bones or on a dressing, in order to specify and/or to check the positioning of the instruments or implants relative to the patient/patient bone and in particular relative to the surgical area with a high degree of accuracy. Known systems and solutions are usually based on external mechanical rods or plates for checking the positioning and alignment of the instruments.

Another disadvantage is that some information cannot be visualized with conventional technology. In particular, there is no way to visualize an axis or section plane directly on the patient's bone. Current instrument systems do not provide reliable control over the exact positioning of the instrument in the surgical field.

From US 2018/0168826 A1, a medical-technology positioning and/or alignment device according to the preamble of claim 1 is known.

### SUMMARY

Against this background, the present invention is based on the object of reducing the aforementioned disadvantages of the prior art, in particular to create a device with which the user-friendliness in positioning surgical instruments, in particular a bone saw or implants, can be maximized with the greatest possible ergonomics and without losses in quality.

Accordingly, the core of the present invention consists in providing a so-called template or guide template, which has to be positioned, aligned and temporarily fixed to the patient or the corresponding patient bone, for example, and which has a guide portion for guiding a surgical instrument (or implant) in accordance with the position and alignment of the template relative to the patient bone. The guide portion is preferably adapted to the surgical instrument to be guided and may, for example, be a longitudinal slot in the case of a bone saw and/or a through-hole in the case of a bone drill or a milling cutter as a surgical instrument, wherein the corresponding guide portion is formed on/in the template in each case.

Furthermore, in the positioning/alignment device according to the invention, an optical marking device (sighting mechanism) is provided, with an (integrated) light source (including power supply), such as e.g. a laser or an LED (the latter if applicable with upstream light aperture, converging lens and/or prism), which (namely the marking device) is designed and adapted to be temporarily fixed (directly and immediately) on/at the template in a predetermined relative position and relative orientation thereto and to project instrument-specific geometric information such as, for example, a cutting line (in the case of, for example, a bone saw) or a drilling axis (in the case of, for example, a bone drill/milling cutter) onto the patient surface.

If the (guide) template is to be fixed to the patient/patient bone, the marking device according to the invention first has to be fixed to the template in the predetermined relative position and its light source has to be activated. Now, the template can be aligned, wherein/while the light source of the marking device projects instrument-specific information, for example a cutting line in the case of a template adapted to a bone saw, onto the surface of the patient/patient bone in the operating area.

Once the template has been correctly placed, aligned and fixed to the patient bone in this way, the marking device can be removed.

In the context of the invention, the instrument-specific geometric information may in particular be, for example, a point and/or a line and/or a plane and/or a three-dimensional representation or hologram.

In the field of orthopedics, prior to the present invention, there were no solutions with regard to direct visualization/projection of such geometric information, in particular of lines and planes, on site, i.e. directly at/on the patient during an operation. A medical technology instrument in the sense of the invention is, as already previously indicated, to be understood in particular as a medical-technology processing instrument such as, for example, a drill, a saw, a milling cutter, a planer, a cutting/welding laser, etc. However, it is also possible to align an implant, in particular an endoprosthesis, with the device according to the invention. Furthermore, in particular geometric information of the patient, such as bone axes or joint axes, start and end points of bones, pivot points of joints, etc., and/or geometric information of the object, such as in particular working directions or axes of a surgical instrument, can also be displayed as visualized/projected geometric information.

Using the invention, one or more planes and axes can be visualized/projected simultaneously or offset in time and directly within the surgical field. In this way, the position and/or alignment of the instruments and the position and/or alignment of the incision guide in the case of a bone saw and thus, for example, a bone incision can be displayed particularly easily and clearly on the patient/patient bone to a surgeon or operator. In the context of the invention, for example, a distal tibia cut can be displayed on the patient/patient bone in relation to the tibia axis. With the positioning and/or alignment device according to the invention, the surgeon is easily able to perceive the influence of malalignments such as varus or valgus and the inclination alignment on the bone section with respect to the frontal and sagittal tibial axis. Furthermore, the femoral frontal axis can additionally be taken into account.

The projection or visualization of the geometric information can be performed by the invention directly at/on the patient, in particular on a bony surface. The visualization/projection of real axes and planes by the optical marking device enables surgeons to more accurately determine the position and alignment of the particular instruments or implants used, thereby avoiding at the same time some inaccurate judgements that could lead to suboptimal decisions.

The idea underlying the invention can be used in almost any operation in which alignment of a (guide) template and an instrument/implant guided by it is performed using hard structures such as bones, for example in operations of the hip, knee and shoulder, in ankle prostheses, osteotomies and also in traumatology.

The idea proposed by the invention takes visual control of instrument alignments and positioning a step further by allowing visualization/projection of axis lines or section planes directly on the patient. It is a particular advantage of the invention that it can be directly integrated into an integrated quality system.

An essential advantage of the invention is the possibility of fast and accurate verification of axes, positions and alignments via direct visualization on patient structures, such as extremities, bones, etc., wherein the inaccuracy and the need for mounting of mechanical rods or control plates, inherent so far in known methods and devices, in particular in a visual assessment, can be avoided. Other advantages include an increase in accuracy, easier implementation of the surgeon's strategy, time savings, and usability for a variety of applications and with different instruments and instrument sets in orthopedics.

An additional benefit is a reduction in the number of alignment and control instruments required for a particular application. Depending on the application, between two and ten instruments may be saved. Using a smaller number of instruments is linked to a lower risk of infection. In to addition to an improvement in quality control, the preparation time/use time/reprocessing time of the instruments before/during/after each operation can also be reduced. This means a gain in operation time, circulation time and/or reprocessing time in central instrument reprocessing.

Considering that currently the only alternatives are pure visual controls without or with the aid of mounted mechanical instruments such as rods and/or control plates, the invention has the advantage of visualizing/projecting axes and/or resection planes directly on patient structures in the surgical field. It is also important to consider that the invention benefits visualization on wrapped and/or uneven surfaces when mental projection is required with the risk of parallax error when using certain instruments.

It is a particular advantage of the invention that the device, by using the optical marking device (sighting mechanism), has a particularly small space requirement, only a few parts susceptible to contamination, and an accuracy that is essentially independent of size. It is therefore particularly accurate, handy, reliable and inexpensive, both in the context of its use and in the context of reprocessing.

Advantageous embodiments of the invention are claimed in the dependent claims and are explained in more detail below.

Preferably, the optical marking device is directly and immediately attached/fixed or attachable/fixable to the guide template. In other words, the optical marking device (a portion thereof) is preferably configured/adapted to be brought into engagement/operative engagement with a portion of the guide template. Accordingly, the guide template (a portion thereof) is preferably configured/adapted to be brought into engagement/operative engagement with a portion of the optical marking device. Direct/immediate fastening/fixing thus means in particular that no further components are provided between the optical marking device and the guide template. Thus, when the medical positioning and/or alignment device of the present disclosure is used as intended, a portion of the component/part ‘optical marking device’ is preferably brought into engagement/operative engagement with a portion of the component/part ‘guide template’, so that the optical marking device is (temporarily) fixed at/on the guide template.

One embodiment is characterized in that the optical sighting mechanism has at least one laser. This can be designed in particular as a laser scanner. According to a further embodiment, the laser may in particular be designed as a line laser. This is to be understood as a laser with special optics with which a line is generated instead of a point when the laser beam strikes an object. This can be achieved, for example, by one-dimensional optical expansion of the laser beam or by rapid oscillation of the laser beam. By using a laser, a particularly precise indication of the position and orientation of the (guide) template and/or of anatomical axes and/or directions relevant for a machining operation can be achieved. In addition, a laser requires only a small installation space, so that the device can be designed to be particularly small, which allows a wide range of applications and a high degree of flexibility. In this case, the surgical field is not covered by the marking device, but is visible and accessible to the surgeon almost without restriction.

A further embodiment of the invention is characterized in that the template is a processing template for guiding a medical-technology processing instrument, in particular a sawing template, a drilling template, or a milling template. In the context of the invention, the template may be designed like any known processing template from medical technology, possibly with the difference that it preferably has a coupling structure for position-determined coupling with the optical marking unit. Within the scope of the invention, the template can be used to align any medical-technology processing instruments and, in particular, to guide them during processing. Examples of such instruments are drills, saws, milling cutters, planers, lasers, etc.

Preferably, the coupling structure consists of at least one recess, in particular in the form of a through hole or blind hole of, for example, round, triangular, quadrangular or polygonal cross-section and/or in the form of a correspondingly shaped projection, in particular a pin. This coupling structure of the template is designed and intended for coupling, engaging and interacting with a suitably shaped coupling structure of the optical marking device. It is of particular advantage if the marking unit is detachably coupled to the template on the user side, since this enables particularly convenient and user-friendly processing by first arranging the template on the patient with the aid of the marking unit coupled thereto in a determined position, aligning it in the intended manner and subsequently fixing it, and then uncoupling and removing the marking device from the (fixed) template for the processing to be carried out.

As an alternative to the aforementioned coupling structure, it is also possible to use the existing guide portions on the template as the template-side coupling structure for the marking device, which has the advantage that the marking device can always be attached to the template in the correct alignment and positioning.

For example, in the case of a guide template adapted to bone saws with a saw blade guide slot, the marking device can have a housing with a sword-like or fin-like coupling structure that can be pressed into the guide slot of the template according to the tongue-and-groove principle. If the fin-like coupling structure is also designed in such a way that it narrows from the fin root (close to the device housing) to its free fin edge, it can be inserted into the guide slot of the marking template in a clamping manner—even in the case of guide slots for different saw blade thicknesses.

In the case of a drilling template (not shown in more detail), the drill hole formed in it can serve as a coupling structure for the marking device, which in this case would be formed with a mandrel-like extension, for example.

According to an embodiment of the invention, the optical marking unit can emit at least a first laser beam or line laser beam. In particular, it can be coupled to the template in such a way that the first laser beam or line laser beam is directed in a machining direction of the template. In this way, the surgeon can align the template and thus the surgical instrument to be used with it particularly easily, quickly and precisely in the manner, position, and direction desired by him.

According to another embodiment of the invention, the optical marking unit may emit at least a second laser beam or line laser beam. In particular, it can be coupled to the template in such a way that the second laser beam or line laser beam is directed in an anatomical axis or direction. This facilitates patient-specific orientation for the surgeon.

In particular, the marking device may be designed to emit the first and possibly the second laser beam/line laser beam. Preferably, the first laser beam/line laser beam and, if applicable, the second laser beam/line laser beam are non-parallel laser beams and are arranged at an angle to each other, in particular aligned orthogonally to each other.

In summary, the invention enables use of line lasers for visualizing/projecting axes, cutting lines and orientation guides directly on the patient. Materialization/visualization of geometric information directly on, for example, a bony surface or visualization of real axes and planes through an optical system (the marking device) can help surgeons to more accurately assess the orientation and alignment of the devices used while avoiding inaccurate judgements leading to suboptimal decisions.

### BRIEF DESCRIPTION OF THE DRAWING FIGURES

Further features and advantages of the present invention will be apparent from the following exemplary and non-limiting description of the invention with reference to figures. These are merely schematic in nature and serve only to aid understanding of the invention. The following is shown:

FIG. 1 shows a perspective view of an embodiment of a device according to the invention,

FIG. 2 shows a side view of the embodiment of FIG. 1,

FIG. 3 shows a perspective view of the embodiment of FIG. 1 from an oblique top view,

FIG. 4 shows a perspective view of the embodiment of FIG. 1 from an oblique bottom view,

FIG. 5 shows a perspective view of a marking unit of the device of FIG. 1 without template,

FIG. 6 shows the marking unit of FIG. 4 from a side view,

FIG. 7 shows the marking unit of FIG. 4 from an oblique top view, and

FIG. 8 shows the marking unit of FIG. 4 from an oblique bottom view.

### DETAILED DESCRIPTION

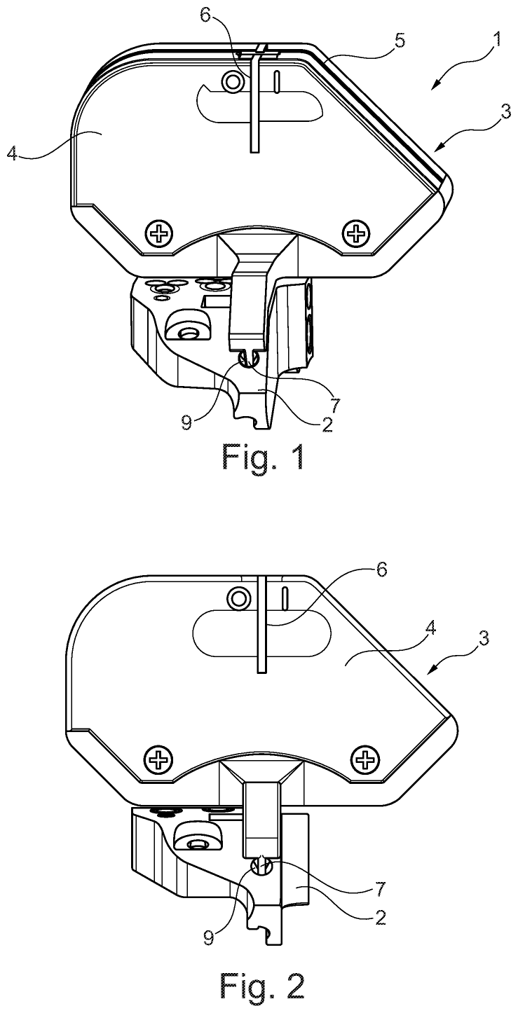

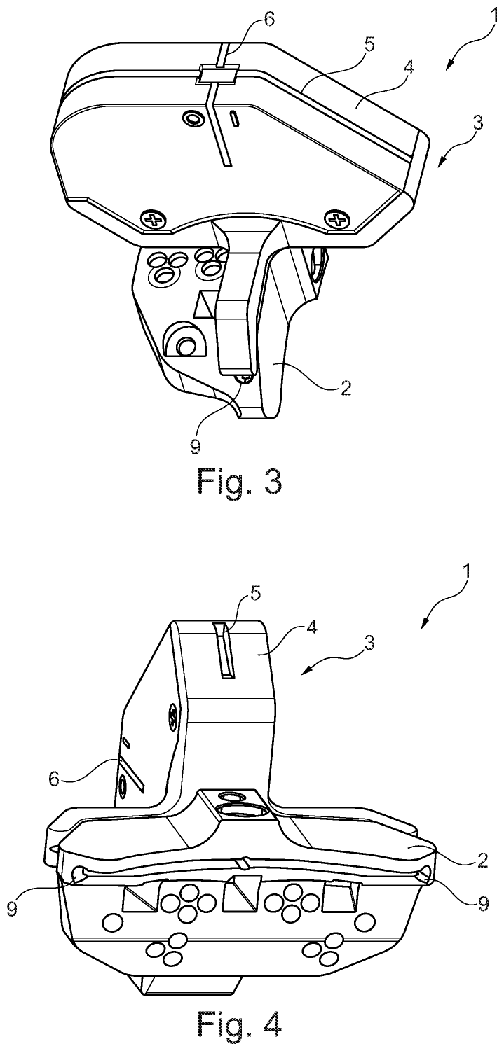

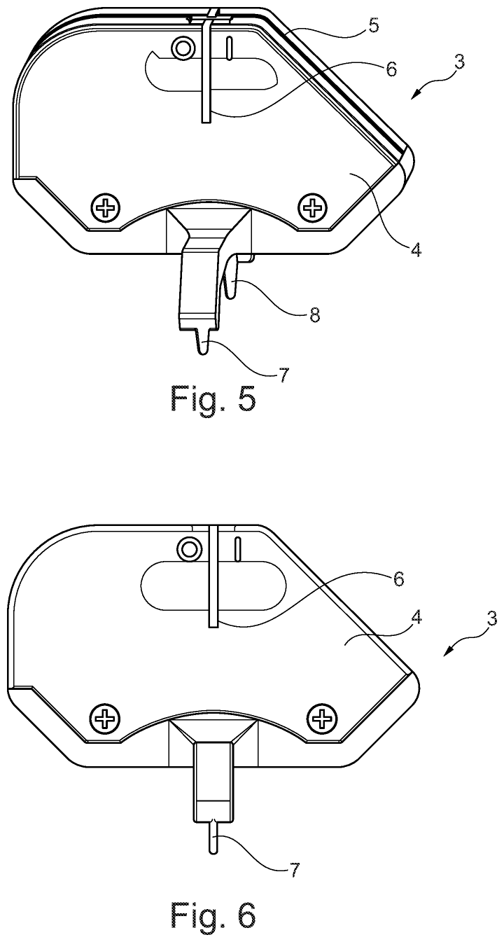

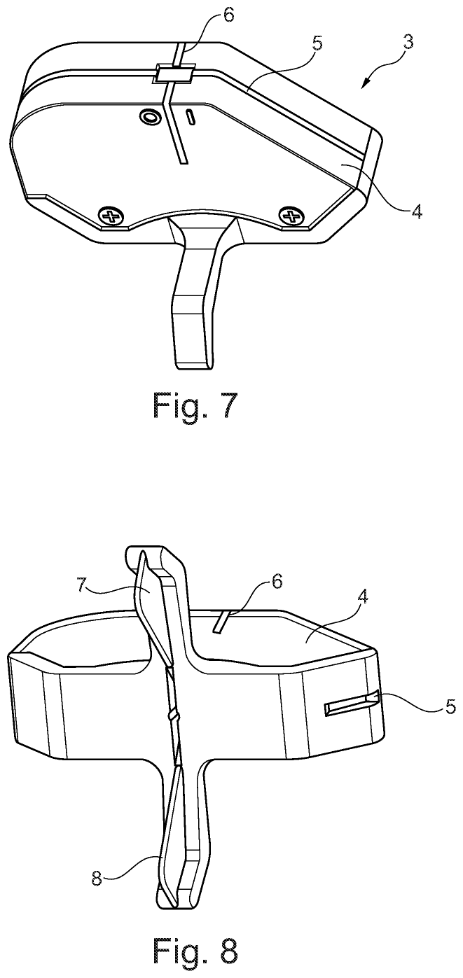

FIGS. 1 to 4 each show a complete medical technology (positioning/aligning) device 1 according to the invention for positioning and/or orienting a surgical instrument or implant not shown in the figures relative to a patient/patient bone also not shown, with direct visualization/projection of instrument/implant-specific geometric information suitable for positioning and/or orienting the instrument/implant on the patient/patient bone. Specifically, FIGS. 1-4 show a sawing template 2 (as already known from the prior art per se) and a marking device 3 already temporarily fixed thereto as a preferred configuration example of the present invention.

Accordingly, the device 1 comprises a (guide) template 2 that interacts or is suitable for interacting with the bone saw to be positioned and/or oriented and an optical marking device/unit 3 (also referred to as sighting mechanism 3) coupled to the template 2 in a determined position for visualizing/projecting saw blade-specific geometric information, for example a cutting line in this case.

The optical sighting mechanism 3 has a line laser that is not visible in the figures and that is arranged in a housing 4 of the marking device 3. This is to be understood as a laser, preferably with special optics, with which a line is generated instead of a dot when the laser beam strikes an object (e.g. a patient bone). In the present example, the optical marking unit 3 emits a first line laser beam 5 and preferably a second line laser beam 6, whose propagation planes are shown on the housing 4 in the figures. The first line laser beam 5 and the second line laser beam 6 are not parallel and are arranged and aligned at an orthogonal angle to each other.

The marking device 3 is coupled to the template 2 in such a way that the first line laser beam 5 is directed in the machining direction of the template 2. The second line laser beam 6 is directed in an anatomical axis or direction. On the one hand, in this way, the position and/or alignment of the instrument used and to be coupled with the template 2, and on the other hand, the position and/or alignment of an incision guide and thus, for example, a bone incision, can be displayed to a surgeon or operator in a particularly simple and clear manner immediately/directly on the patient (bone). For example, a distal tibia cut can be displayed in relation to the tibia axis, wherein the first line laser beam 5 marks and visualizes the cutting direction and the second line laser beam 6 marks and visualizes the tibia axis.

For position-determined coupling with the template 2, the marking unit 3 has a coupling structure according to the invention, in this case in the form of a first projection 7 and a second projection 8 spaced apart from it. The template 2 has a recess 9 matching the projections 7, 8 in the form of a through opening 9. The marking unit 3 can be easily inserted into the template and is coupled to it in a determined position when inserted. It can be removed particularly easily by the user by pulling it off the template 2 so that the latter is ready for alignment and guidance of an instrument not shown in the figures.

As can be seen in particular from FIG. 8, the housing 4 of the marking device 3 has (on the underside) a support/mounting bracket or mounting base consisting of a guide rail on which the two-part, in this configuration example sword-like or fin-like projection 7, 8 is formed. I.e. the two sword-like or fin-like projections are arranged parallel to each other on a line. The projections 7, 8 form the marking device-side coupling structure, which is provided to engage with the template-side coupling structure.

Since the (guide) template 2 in the present configuration example is a saw blade template, the template-side coupling structure relates to the saw blade guide slit (generally referred to as through opening 9 above), as it is indicated in FIGS. 1 and 2. Preferably, the fin-like projections 7, 8 can be triangular or trapezoidal in their respective cross-section so that they can be pressed into the guide slit 9 (according to the tongue and groove principle) in order to hold/fix the housing 4 of the marking device 3 frictionally in the guide slit 9 of the template 2.

As can also be seen in particular from FIG. 1, the second laser/laser beam 6 (in this case with a slit-shaped light exit opening) is located immediately above the template-side coupling structure and thus on the same level as the guide slit of the saw blade template 2. In other words, the laser beam and the sword-like or fin-like projections 7, 8 run essentially parallel (possibly converging, but not skewed). In the fixed state of the marking device 3 on the template 2, a line can thus be projected onto the patient/patient bone with the aid of the at least one (second) laser/laser beam 6, said line extending virtually as an extension of the guide slit 9 of the template 2 and thus optically representing a cutting line of the saw blade.

As soon as the template 2 has been fixed in the correct alignment and position on the patient/patient bone, the marking device 3 can be removed from the template 2 and the guide slit or guide groove 9 for the saw blade (not shown) can be released.

Finally, it should be noted that in particular the marking device 3 according to the invention is designed as a single use product. For this purpose, the housing 4 of the marking device 3 including the coupling structure 7, 8 is made of a plastic (e.g. injection molded), whereas the light source (including current source) enclosed in the housing is preferably formed by a laser/laser device. This means that the entire device 3 can be manufactured quickly and inexpensively, so that reprocessing is not justified from an economic point of view.

1. A medical positioning and/or alignment device comprising:
a guide template for a surgical instrument which can be positioned, aligned, and temporarily fixed to a bone and which has a guide portion for guiding the surgical instrument in accordance with a position and alignment of the guide template relative to the bone; and
an optical marking device which has at least one light source,
the optical marking device being designed and adapted:
to be temporarily fixed on/at the guide template in a predetermined relative position and relative orientation thereto, and
to project instrument-specific geometric information in a predetermined relation or positional relationship to the guide portion of the guide template onto the patient,
the optical marking device comprising a housing in which the at least one light source is housed and on which a first coupling structure is formed that is operatively engageable with a second coupling structure on the guide template.

2. (canceled)

3. The medical positioning and/or alignment device according to claim 1, wherein the guide portion has or is a longitudinal slot.

4. The medical positioning and/or alignment device according to claim 1, wherein the guide portion has or is a through hole.

5. (canceled)

6. The medical positioning and/or alignment device according to claim 3, wherein the second coupling structure is the longitudinal slot and the first coupling structure on the optical marking device is a mounting base with a fin-shaped projection formed thereon which is adapted and provided to be frictionally pressed into the longitudinal slot.

7. The medical positioning and/or alignment device according to claim 1, wherein the at least one light source comprises line laser.

8. The medical positioning and/or alignment device according to claim 1, wherein the at least one light source is a laser scanner.

9. The medical positioning and/or alignment device according to claim 1, wherein the optical marking device emits at least a first beam and is coupled to the guide template in such a way that the first beam is directed in a machining direction of the guide template.

10. The medical positioning and/or alignment device according to claim 9, wherein the optical marking device emits at least a second beam and is coupled to the guide template such that the second beam is directed in an anatomical axis or direction.

11. The medical positioning and/or alignment device according to claim 10, wherein the first beam and the second beam are arranged at an angle to each other.

12. A marking device for a medical positioning and/or alignment device according to claim 1 comprising a mounting base on which a fin-like projection is formed, a free fin edge of which is aligned along a radiation direction of the at least one light source.

13. The marking device according to claim 12, wherein the housing and the coupling structure are made of plastic.
